SEQUENCE LISTING (4 sequences)

SEQ ID NO: 1 - protein, 23 aa, Artificial Sequence
  Peptidic Fragment of alpha-laminine
CSRARKQAASIKVAVSADRHHHH

SEQ ID NO: 2 - protein, 31 aa, Artificial Sequence
  N-terminal sequence of the alpha-amphipatic helix of NS5A of the hepatitis C virus
SGSWLRDVWDWICTVLTDFKTWLQSKLDYKD

SEQ ID NO: 3 - protein, 51 aa, Artificial Sequence
  P4H-Hx40
CSRARKRARKRARKRARKRARKRARKRARKRARKQAASIKVAVSADRHHHH

SEQ ID NO: 4 - protein, 71 aa, Artificial Sequence
  P4H-Myo
CIKREPFVAPAGLTPNEIDSTWSALEKAEQEHAEALRIELKRQKKIAVLSRARKQAASIKVAVSADRHHHH

(19) United States
(12) Patent Application Publication
(10) Pub. No.: US 2018/0024125 A1
(43) Pub. Date: Jan. 25, 2018

(54) SUBSTRATE/PEPTIDE/LIPID BILAYER ASSEMBLY, PREPARATION METHODS AND ASSOCIATED DETECTION METHODS

(71) Applicants: UNIVERSITE CLAUDE BERNARD LYON I, VILLEURBANNE CEDEX (FR); CENTRE NATIONAL DE LA RECHERCHE SCIENTIFIQUE (CNRS), PARIS CEDEX 16 (FR); INSTITUT NATIONAL DES SCIENCES APPLIQUEES DE LYON, VILLEURBANNE CEDEX (FR); ECOLE SUPERIEURE DE CHIMIE PHYSIQUE ELECTRONIQUE DE LYON, VILLEURBANNE CEDEX (FR)

(72) Inventors: Agnes GIRARD-EGROT, BEYNOST (FR); Ofelia MANITI, LA TOUR DU PIN (FR); Samuel REBAUD, SAINT-ETIENNE (FR)

(21) Appl. No.: 15/520,896

(22) PCT Filed: Oct. 27, 2015
(86) PCT No.: PCT/FR2015/052890

§ 371 Date: Apr. 21, 2017

(30) Foreign Application Priority Data
Oct. 28, 2014 (FR) 1460349

(51) Int. Cl. G01N 33/543

(52) U.S. Cl. CPC G01N 33/5432

(57) ABSTRACT

The invention concerns an assembly consisting of a substrate on which at least one lipid bilayer is attached by means of a peptide, referred to as the tethering peptide, which is itself linked to the substrate, characterised in that the tethering peptide has a C-terminal end constituted by at least four consecutive histidines and in that the lipid bilayer comprises a portion of lipids having a chelating headgroup enclosing a metal cation providing the link with the tethering peptide as a result of metal-chelate interactions between the metal cation and at least a portion of the histidines located at the C-terminal position of the tethering peptide; and the method for preparing same and the associated detection methods.

The present invention relates to the technical field of lipid membranes attached to an analysis substrate. In particular, the invention relates to a substrate bearing a lipid bilayer, the link between the substrate and the lipid bilayer being established by means of a specific peptide, a method for preparing such a substrate and the use thereof as a biochip and in various analysis techniques.

Membrane proteins play a major role in each living cell. These proteins are the linchpins of cellular metabolism and of biological signal transduction. Because of their important functions, they are preferred therapeutic targets. However, the reconstitution of membrane proteins remains a challenge because it requires a membrane environment in order to be functional. To overcome this problem, biomimetic membranes corresponding to lipid bilayers attached to a substrate are regarded as a leading model for the study of biological membranes in various branches of basic research. At present, the integration of biomimetic membranes incorporating transmembrane proteins into biochip design remains an important challenge for the high-throughput screening of medicinal products and the development of rapid diagnostic tests.

Membrane biochips thus have a promising analytical potential, notably for two principal applications. After reinsertion of membrane proteins of interest (notably a transmembrane receptor) into the membranes present on the zones of analysis (also called “arrays”) of a biochip, it is possible:
either to study the effect of various ligands (notably agonists/antagonists) on the functionality of the reinserted protein;
or to screen the action of a therapeutic molecule on various receptors if various receptors have been inserted on the membrane arrays of the chip.

Despite these applications and the analytical performance that can be associated with the use of membrane biochips, the major obstacle to be overcome for the reconstitution of transmembrane proteins and for the development of membrane biochips resides in the formation in vitro of a robust lipid bilayer offering the capacity to insert integral transmembrane proteins, without altering the intrinsic properties of such proteins. Most lipid bilayers reconstituted in vitro on the surface of substrates, notably of the gold type, are in direct contact with the substrate, i.e., only a thin, two to three nanometers thick layer of water separates the lipid bilayer from the substrate. Also, even if these so-called supported lipid bilayers are easy to handle owing to the fact that they are deposited on a solid substrate, the presence of the latter nearby, with an insufficient gap, prevents the functional reconstitution of transmembrane proteins, due to the presence of often bulky extramembrane ectodomains encountered in these protein structures. Moreover, the insufficient gap between the lipid bilayer and the substrate most often causes a loss of mobility of the reinserted membrane proteins, which may interact directly with the substrate.

To circumvent this problem, various solutions have been proposed in the prior art, aiming to separate the lipid membrane from the substrate and to obtain assemblies consisting of a lipid bilayer attached to the solid substrate by means of a hydrophilic molecular spacer.

To that end, it has been proposed to link the lipid bilayer to the substrate by means of a flexible and hydrophilic molecular layer thus providing a separation between the lipid bilayer and the substrate. Such an intermediate layer also acts as a water reservoir between the substrate and the membrane and provides a sufficient space for increasing the mobility of the reinserted proteins.

Various techniques for manufacturing membranes and for attaching same to substrates for biochips have been proposed in the prior art. Polymer-, thioalkane-, and protein-type spacers have been envisaged. The publication by Coutable et al. 2014 describes, for example, the use of a PEG (polyethylene glycol) to establish the link between the lipid bilayer and the substrate. This technique uses a specific functionalized lipid, namely 1,2-distearoyl-sn-glycero-3-phosphoethanolamine-N-poly-(ethyleneglycol)-2000-N-[3-(2-pyridyldithio)-propionate] (DSPE-PEG-PDP) mixed with POPC (1-palmitoyl-2-oleoyl-sn-glycero-3-phosphocholine). Pyridyldithiopropionate (PDP) allows the formation of an S—Au bond on the gold surface. In this article, only POPC is used in addition to the functionalized lipid. Moreover, DSPE has saturated hydrophobic chains that may influence the fluidity of the lipid membrane obtained. PEG seems also to have an impact on the reinsertion of the membrane protein.

Among the strategies proposed, the use of polar peptides as spacers is regarded as the most promising, to then facilitate the functional reincorporation of membrane proteins. They offer the advantage of constituting a biocompatible medium of the same nature as the protein to be inserted and can play the part of cytoskeleton.

Typically, the peptide tethers or spacers used heretofore consist of: 1) a functional group such as a thiol function, a disulfide or a silane, which can bind covalently to a suitable substrate such as gold, 2) a peptide portion which plays the part of hydrophilic spacer and, 3) a hydrophobic lipid portion which allows the anchoring of the bilayer by forming the proximal leaflet of the reconstituted lipid membrane.

This first proximal membrane leaflet is obtained by self-assembly on the substrate of a dilute solution of spacer peptides comprising a lipid component. The covalent bond with the substrate allows stable tethering of the proximal layer and thus of the bilayer. The distal leaflet of the membrane is then formed, either by deposition of a Langmuir monolayer by Langmuir-Blodgett transfer, or by liposome fusion, directly on the hydrophobic lipid surface of the proximal leaflet formed beforehand on the substrate by self-assembly. The liposome fusion is, in particular, selected to obtain the direct fusion of proteoliposomes and to then promote the reconstitution of membrane proteins in the attached suspended bilayer.

The spacer peptides used as molecular tethers are prepared from natural or synthetic thiopeptides or thiolipopeptides. In Robelek et al. 2007 or Yildiz et al. 2013, the peptides are attached to a gold substrate by means of a lipoic acid or a sulfhydryl group of a cysteine at the N-terminal position and their C-terminal end is then activated to chemically couple a phospholipid, which corresponds to a dimyristoyl-phosphatidyl-ethanolamine (DMPE) molecule by means of the NH₂ function of the polar headgroup of this phospholipid. To that end, the terminal —COOH group of the thiopeptides is activated by N-ethyl-N′-(dimethylaminopropyl)carbodiimide/N-hydroxysuccinimide (EDC/NHS) to form a covalent ester bond with the NH₂ function of DMPE. However, this method demands that the proximal leaflet of the bilayer consists chiefly, even only, of DMPE. It is thus not possible, in this case, to vary the lipid composition of the latter, which can be a major disadvantage during the reconstitution of the membrane protein of interest. Indeed, it is well-known that all biological membranes (plasma, nuclear, mitochondrial, chloroplast, etc.) have different compositions, in terms of the nature of the lipids and their proportions. Moreover, in the solutions of the prior art, the lipid bilayers are anchored to their proximal leaflet only by hydrophobic interaction, by means of a cholesterol core or of saturated hydrophobic acid chains (notably fatty chains of DMPE attached to the C-terminal end of the spacer peptide) which penetrate the liposome during the formation of the distal leaflet. This mode of interaction may introduce local disruptions within the lipid bilayer during its formation, which may lead to partial destruction of the membrane architecture during handling of the membrane in aqueous medium.

Also, in the prior art, the proximal layer is first constructed by means of a process of self-assembly of a dilute solution of the tether molecules which generally correspond to thiolipopeptides, i.e., to peptides functionalized by a hydrophobic lipid domain. The distal layer of the membrane is then formed, either by liposome fusion, or by Langmuir-Blodgett deposition on the self-assembled hydrophobic monolayer. In this case, even if the anchoring of the hydrophobic proximal layer makes it possible to enhance the mechanical stability of the lipid membrane obtained in relation to a supported lipid bilayer only adsorbed onto the substrate, this two-step formation procedure guarantees neither the formation of a planar and continuous bilayer, nor the versatility of the lipid composition of the membrane, as the presence of the hydrophobic lipid layer due to the anchoring method used limits the choice of the composition of the proximal leaflet. Moreover, the synthetic chemistry of the peptides used to obtain the first leaflet comprising the hydrophobic lipid can be complex and the dynamic behavior of the lipid bilayer anchored by its proximal leaflet may be modified. The formation of the two leaflets of the bilayer in two distinct steps is thus a major disadvantage for the reconstitution of integral transmembrane proteins which require a uniform lipid environment in order to retain their functionalities.

Technological advances concerning the manufacture and the industrial applications of membrane biochips dedicated to the reconstitution of transmembrane proteins thus remain very limited.

The objective that the present invention proposes to solve is to propose a solution which is suited to the attachment of a wide range of lipids and, thus, to the tethering of various lipid bilayers compatible with the insertion of an extensive range of proteins, so as notably to propose new solutions suited to the development of biochips.

Another objective of invention is to propose a method which is both simple and versatile, which allows the formation of lipid bilayers attached to a substrate, wherein the link between the lipid bilayer and the substrate must be robust and suited to various lipid compositions, so as to be able to vary and to adjust as desired the composition of the attached lipid bilayer and thus to allow the incorporation of membrane proteins, and notably of integral membrane proteins, without altering their intrinsic properties.

In this context, the invention relates to an assembly consisting of a substrate on which at least one lipid bilayer is attached by means of a peptide, referred to as the tethering peptide, which is itself linked to the substrate, characterized in that the tethering peptide has a C-terminal end consisting of at least four consecutive histidines and in that the lipid bilayer comprises a portion of lipids having a chelating polar headgroup enclosing a metal cation providing the link with the tethering peptide as a result of metal-chelate interactions between the metal cation and at least a portion of the histidines constituting the C-terminal end of the tethering peptide.

In the context of the invention, the mode of linkage used to establish the link between the tethering peptide and the lipid bilayer makes it possible to adjust as desired the composition of the lipid bilayer which may correspond to any cell membrane type, given that, in the context of the invention, this composition is no longer conditioned by a spacer peptide functionalized by a hydrophobic lipid domain which would be used to form the proximal lipid layer (i.e., that located near to the substrate), but by the nature of the lipids which would be attached thereto. In the context of the invention, the lipid bilayer is attached to the tethering peptide which serves as a spacer as a result of a metal-chelate interaction established between the metal ion and the imidazole ring of at least some of the histidines forming the C-terminal end of the tethering peptide. Some of the imidazoles of the polyhistidine end of the tethering peptide thus also play the role of chelate for the metal ion present, like the chelating portion present on the lipid having a chelating headgroup. The link thus established gives the assembly high stability and high robustness which are superior to those obtained with the solutions of the prior art, thus allowing it to be handled.

The lipid bilayer is thus attached to the substrate by means of the tethering peptide, while being kept at a distance from the substrate, and may be regarded as being suspended due to the flexibility of the tethering peptides.

Various chelating lipids enclosing a metal cation are available commercially, and, for example, marketed by the company Avanti Polar Lipids (Alabaster, Ala., United States) and may be used in the context of the invention. Mention may be made of 1,2-dimyristoyl-sn-glycero-3-phosphoethanolamine-N-diethylenetriamine pentaacetic acid (14:0 PE-DTPA) gadolinium salt or copper salt, 1,2-dipalmitoyl-sn-glycero-3-phosphoethanolamine-N-diethylenetriaminepentaacetic acid (16:0 PE-DTPA) gadolinium salt or copper salt, 1,2-distearoyl-sn-glycero-3-phosphoethanolamine-N-diethylenetriaminepentaacetic acid (18:0 PE-DTPA) gadolinium salt or copper salt, 1,2-dioleoyl-sn-glycero-3-[(N-(5-amino-1-carboxypentyl)iminodiacetic acid)succinyl] (DGS-NTA) nickel salt, DTPA-bis(stearylamide) gadolinium salt, bis(1,2-dimyristoyl-sn-glycero-3-phosphoethanolamine)-N—N′-diethylenetriaminepentaacetic acid (bis(14:0 PE)-DTPA) gadolinium salt, bis(1,2-dipalmitoyl-sn-glycero-3-phosphoethanolamine)-N—N′-diethylenetriaminepentaacetic acid (bis(16:0 PE)-DTPA) gadolinium salt, bis(1,2-distearoyl-sn-glycero-3-phosphoethanolamine)-N—N′-diethylenetriaminepentaacetic acid (bis(18:0 PE)-DTPA) gadolinium salt.

In the context of the invention, the metal cation is, for example, a nickel cation (Ni²⁺), a gadolinium cation (Gd³⁺) or a copper cation (Cu²⁺). The nitrilotriacetic (NTA) group trapping a nickel cation is the most effective for attaching the tethering peptide by means of its polyhistidine end. Also, preferably, the metal cation is a nickel cation and the chelating polar headgroup is nitrilotriacetic acid.

The C-terminal end of the tethering peptide, in turn, consists, for example, of four, five or six consecutive histidines. Interactions will be established between the metal cation and at least some of the imidazole groups of the histidines located at the C-terminal end of the tethering peptide. More precisely, a metal-chelate-type bond is established with the nitrogen bearing a free electron pair of at least some of the imidazole groups of the histidines, such as for example illustrated in Scheme 1 below in the case of Ni-NTA:

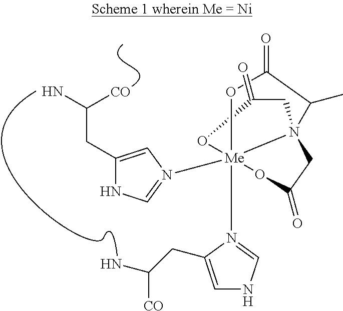

Preferably, the link with the tethering peptide is provided as a result of metal-chelate-type interactions established between the metal cation and two of the histidines located in the C-terminal portion of the tethering peptide.

In the context of the invention, the portion of lipids having a chelating polar headgroup enclosing a metal cation is small and in no way alters the properties of the lipid bilayer which can thus mimic, as closely as possible, the composition of biological membranes, and notably of cell membranes. Advantageously, the portion of lipids having a chelating polar headgroup enclosing a metal cation represents from 0.5 to 5 mol %, preferably from 1 to 2 mol %, of the totality of the lipids forming the lipid bilayer. The lipid(s) having a chelating polar headgroup enclosing a metal cation is(are) distributed in the lipid bilayer and provide(s) the anchoring of the bilayer, with a good distribution within the latter.

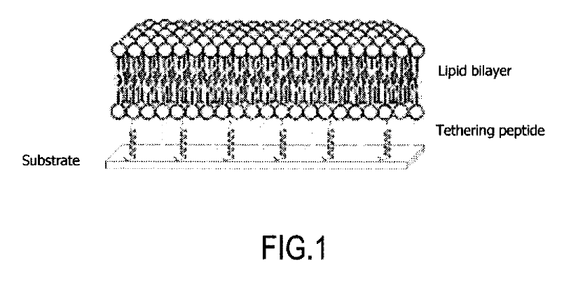

Traditionally, by “lipid bilayer” is meant a double lipid layer, consisting of two leaflets of lipid molecules of which the majority consist of phospholipids, so as to allow such a leaflet structure. Such a bilayer constitutes a thin polar membrane. Phospholipids comprise a polar headgroup and at least two aliphatic chains. The polar headgroups make up the membrane surface and the aliphatic chains are oriented towards the interior of the membrane, as illustrated in FIG. 1.

By “phospholipids” is meant glycerophospholipids, including phosphoinositides and sphingophospholipids. The lipid bilayer will consist, preferably, of at least 70% by mass of phospholipids, preferably of at least 80% by mass and preferentially of at least 90% by mass, indeed of 100% by mass of phospholipids. There is no reason that the lipid bilayer cannot include other natural lipids, for example, such as cholesterol. Such lipids will be present in a quantity such that it does not affect structuring in the form of a bilayer.

In the context of the invention, the lipid composition of the bilayer may be adjusted as desired, because it is independent of the bilayer formation protocol. Many lipid compositions may be used, and in various proportions, which makes it possible to mimic in a tailored manner the composition of the various types of biological membranes. Excluding the portion of the lipids which has a chelating polar headgroup enclosing a metal cation, the lipid bilayers may consist of any lipid alone or as a mixture, including but not limited to, phosphatidylcholine (PC), phosphatidylserine (PS), phosphatidylethanolamine (PE), phosphatidylinositol (PI), phosphatidylinositol bisphosphate (PIP₂), cholesterol, sphingomyelin. Lipid bilayers of variable compositions including the various classes of membrane lipids such as phospholipids (including phosphoinositides), sphingolipids and cholesterol can be obtained. The lipid bilayers may also be formed from natural extracts of membrane lipids. The possibility of specifically adapting the composition of the lipid bilayer, so as to obtain an artificial membrane for the protein which one wishes to insert, makes it possible to retain its natural lipid environment and then to guarantee its reinsertion.

Advantageously, the composition of the lipid bilayer is selected so as to mimic a biological membrane, and notably a cell membrane, in particular plasma, nuclear, mitochondrial, chloroplast, etc. In the context of the invention, it is possible to obtain an assembly whose lipid bilayer is fluid and continuous. To that end, the lipid bilayer will be formed preferably of at least 80%, preferably of at least 90%, and preferably of at least 95% by mass of fluid lipids. The fluidity of a lipid is defined in terms of the order of the fatty acyl chains at room temperature (22° C. notably). A lipid is said to be fluid when the arrangement of its fatty chains is disrupted (i.e., “gauche” conformations on a chemical level). The fluidity of a given lipid depends mainly on the length of its acyl chains, their unsaturation and the temperature. By way of example, at room temperature, phospholipids having unsaturated acyl chains (C18:1, for example) are in the fluid state.

With the mode of linkage proposed in the context of the invention, a certain variability of the lipid composition is possible: it is notably possible to select the acyl chains, the length of the chain, to select an acyl chain with one or more unsaturations.

So as to guarantee the permanent attachment of the lipid bilayer and the robustness of the assembly, the tethering peptide is, preferably, attached covalently to the substrate, preferably by its N-terminal end. High-affinity interactions, notably of the biotin-streptavidin type, may also be envisaged.

Various immobilization strategies by covalent bond may be implemented, as a function of the nature of the substrate. It is possible to use the techniques implemented in the prior art. A covalent bond may, for example, be established by reaction of an amine function of the peptide: an amine function of the amino acid located at the N-terminal position or an amine function of the side chain of a lysine. It is also possible to establish a covalent bond by reaction with the thiol function of a cysteine or by insertion of a disulfide or a lipoic acid at the N-terminal end of the peptide.

As a function of the nature of the substrate, the reaction for establishing a covalent bond may require a preliminary functionalization of the substrate. This will be the case notably when the substrate is made of glass, of polymer, of silanized glass. For example, the thiol function of a cysteine can react with a surface functionalized by a chemical group such as maleimide. An amine function can, in turn, establish a covalent bond with a functional group such as aldehydes, activated esters, epoxies. Such groups may be introduced beforehand onto the substrate, notably onto silanized glass slides, according to techniques well-known to the person skilled in the art.

In the case of a gold substrate, a covalent bond can be established directly with a thiol function, by formation of an S—Au bond by chemisorption. Moreover, gold substrates are the substrates of choice for detecting molecular interactions by quartz crystal microbalance with dissipation monitoring (QCM-D) and by SARI. Also, preferably, in the context of the invention, the substrate is made of gold. In this case, advantageously, the tethering peptide comprises a cysteine at its N-terminal end establishing an S—Au bond with the gold substrate.

Preferably, the peptide will have a hydrophilic structure to prevent its adhesion onto the substrate. The conformation and the structure of the tethering peptide, the hydrophilic properties and the length thereof can be controlled by the nature and the number of amino acid residues comprising said peptide. These characteristics may be adjusted by the person skilled in the art, so as to obtain the desired gap between the lipid bilayer and to control the viscosity of the intermediate layer constituted by the tethering peptide in order to provide the lateral fluidity necessary for the functional reinsertion of transmembrane proteins. In addition to the amino acids of the N- and C-terminal ends described above, the peptide advantageously will consist of amino acids selected from hydrophilic amino acids, such as lysine, arginine, glutamic acid, aspartic acid, asparagine, glutamine and serine. In the context of the invention, use may be made, for example, of the peptide of SEQ ID NO: 1: CSRARKQAASIKVAVSADRHHHH or one of the following peptides of SEQ ID NO: 3: CSRARKRARKRARKRARKRARKRARKRARKRARKQAASIKVAVSADRHHHH or of SEQ ID NO: 4: CIKREPFVAPAGLTPNEIDSTWSALEKAEQEHAEALRIELKRQKKIAVLSRARKQAASI KVAVSADRHHHH. The tethering peptide of SEQ ID NO: 1 was synthesized on the basis of a peptide fragment derived from the α-laminin (P19) having a cysteine at the N-terminal position for grafting onto gold and a polyhistidine tag at the C-terminal position for attaching liposomes by metal-chelate-type bonds.

Ideally, if it is possible to know if the protein of interest to be reinserted into the lipid bilayer has preferred interactions with cytoskeletal proteins and to know the sequence which would be responsible for said interactions, the latter may be incorporated into the sequence of the tethering peptide in order to promote the interactions with the protein and to stabilize its conformation after reinsertion.

The quantity of tethering peptide used will be selected so as to establish a sufficient attachment. For example, in the case of a gold substrate, the tethering peptide of SEQ ID NO: 1 is present with a density of 2.8·10¹³ molecules per cm², corresponding to saturation of the surface of the gold substrate. The peptide will be distributed and attached homogeneously to the substrate surface.

The selected length of the peptide will be adapted by the person skilled in the art, notably as a function of the size of the ectodomain of the protein to be reinserted. If the substrate is made of gold, it will also be necessary to take into account the sensitivity of the SPRi apparatus which will be used for the subsequent analysis, so as not to lose the signal in the event of interactions too distant from the substrate surface.

Generally, the peptides used heretofore in the literature generate a space of 2 nm. A space of 6 nm is ideal for reconstituting a wide range of membrane proteins (Schiller S. M., Reisinger-Friebis A., Götz H., Hawker C. J., Frank C. W., Naumann R., and Knoll W., Biomimetic Lipoglycopolymer Membranes: Photochemical Surface Attachment of Supramolecular Architectures with Defined Orientation. Angewandte Chemie International Edition, 2009, 48(37): 6896). Also, in the context of the invention, the peptide will be selected preferably so as to establish a spacing between the substrate and the lipid bilayer of 1 to 10 nm, and preferentially of 2 to 7 nm, and in particular of about 6 nm.

In the context of the invention, the assembly may contain a membrane protein, preferably an integral membrane protein, which is inserted into the lipid bilayer. A protein is said to be integral when it crosses at least once completely a lipid bilayer of a cell membrane. As explained below, the membrane protein may be introduced after the formation of the lipid bilayer or directly during the formation of the latter.

The assemblies, according to the invention, may be used for forming biochips. In particular, it is possible to express membrane proteins in a lipid bilayer mimicking a lipid membrane, and this in an acellular manner. As a function of the nature of the membrane protein of interest which will be inserted, the substrate may be used as an analysis tool, notably for studying or for screening new agonists or antagonists or for studying new therapeutic agents directed at membranes. The use of a gold substrate will make it possible, in particular, to carry out measurements by surface plasmon resonance imaging (SPRi), which requires no preliminary labeling of the molecular entities to be studied.

So as to be able to be used as biochips, the assembly according to the invention will comprise a substrate which has several zones on which a lipid bilayer is attached by means of a peptide, referred to as the tethering peptide, which is itself linked to the substrate, said tethering peptide having a C-terminal end consisting of at least four consecutive histidines and the lipid bilayer comprising a portion of lipids having a chelating polar headgroup which encloses a metal cation and provides the link with the tethering peptide as a result of metal-chelate-type interactions between the metal cation and at least a portion of the histidines located in the C-terminal portion of the tethering peptide.

In this case, the assembly corresponds to a biochip. Preferably, at least two, indeed more, of the zones which will correspond to different zones of analysis or spots, bear a different lipid bilayer, making it possible to carry out different analyses simultaneously. The various features mentioned in the context of the invention, in relation to the definition of the tethering peptide, of the bilayer membrane, and of the method for preparing the substrate/tethering peptide/lipid bilayer assemblies, apply to each one of these zones.

The invention also has as an object a method of detection by surface plasmon resonance imaging using an assembly according to the invention wherein the substrate is made of gold.

The invention also relates to a method for preparing an assembly according to the invention which comprises the following successive steps:
a) attaching the tethering peptide to the surface of the substrate,
b) attaching onto the tethering peptide liposomes comprising a portion of lipids having a chelating polar headgroup enclosing a metal cation, said attaching being carried out by the establishment of metal-chelate interactions between the metal cation and at least a portion of the histidines located in the C-terminal portion;
c) adding a fusogenic agent in order to induce liposome fusion and the formation of a continuous lipid bilayer.

In the context of the invention, the mode of attachment of the lipid bilayer onto the substrate is compatible with various compositions of bilipid membranes which may thus be adjusted, as a function of the transmembrane protein to be reinserted.

In the method according to the invention, the two leaflets of the lipid bilayer are formed simultaneously, unlike the protocols described in the prior art, wherein the two leaflets were formed independently in two distinct steps. In the context of the invention, the method is simplified, since the formation of the two leaflets of the bilayer is carried out in a single step. The lipid bilayers obtained retain their dynamic behavior and their fluidity since the bilayer is not anchored by its proximal leaflet and since it is attached to the tethering peptide only by a few percent (in particular from 0.5 to 5 mol %, and preferably from 1 to 2 mol %) of the lipids having a chelating headgroup included in the composition of the liposomes. Hence, the corresponding lipid bilayers can be easily used for the reinsertion of membrane proteins, by acellular expression in vitro of the protein in the presence of the planar and continuous lipid bilayer obtained.

Another advantage of the mode of linkage used in the context of the invention, which employs lipids with a chelating headgroup enclosing a metal cation establishing metal-chelate-type bonds with the histidines, is that these interactions are reversible. Indeed, it is possible to regenerate the surface by removing the lipid bilayer and to re-use the substrate: i.e., after insertion of a protein and use, it is possible to remove the lipid bilayer in order to keep only the intermediate substrate functionalized with the tethering peptide, which is thus also an integral part of the invention. In the case of industrial application, this regeneration will ultimately lead to a considerable reduction in the costs of producing biochips and of large-scale sample analysis.

The invention thus also has as an object an intermediate substrate on which a peptide, referred to as the tethering peptide, is attached, preferably by its N-terminal end, characterized in that the tethering peptide has a free C-terminal end consisting of at least four consecutive histidines. The characteristics of the substrate and of the peptide described in the context of the invention apply to this intermediate substrate.

In the context of the invention, the lipid bilayer is, in most cases, obtained by fusion of unilamellar liposomes having an average diameter ranging between 30 and 500 nm. The average diameter of the liposomes may be defined as the number-average diameter of the liposome population used, determined by quasi-elastic light scattering, also called dynamic light scattering (DLS).

By “unilamellar liposome” is meant a liposome which consists of only one lipid bilayer.

Such a liposome fusion technique produces an integral, homogeneous, planar and continuous layer. The liposomes are not broken up during their attachment to the tethering peptide, and remain intact. It is then the action of the fusogenic peptide which brings about the rupture of the liposome and the formation of the bilayer. The opening of the liposome and the formation of the bilayer are known by the term “liposome fusion”.

Fusion is, preferably, obtained thanks to a fusogenic peptide, preferably of SEQ ID NO: 2: SGSWLRDVWDWICTVLTDFKTWLQSKLDYKD. This peptide corresponds to the 31 amino acids of the N-terminal sequence of the amphipathic alpha-helix (AH) of the hepatitis C virus nonstructural protein (NS5A) necessary to the membrane association of the virus during its replication.

Such a fusogenic peptide has been described and used notably in the publications by Cho et al. 2007, 2009 and 2012, by Coutable et al. 2014 and in the U.S. Pat. No. 8,211,712, for the fusion of liposomes of various compositions. This peptide is removed and is not found in the final assembly (Hardy et at 2012). Moreover, the publication by Coutable et al showed that the use of this fusogenic peptide is compatible with the reinsertion of membrane proteins.

Other fusogenic peptides may be envisaged: peptides derived from viral capsid proteins, such as hemagglutinin (HA2), gp41 (HIV), gp 30, gp 32, P15E (murine leukemia virus). For more details concerning such peptides, reference may be made to A. Lorin et al 2007.

In the context of the invention, it is possible that the liposomes are proteoliposomes. In this case, the insertion of the protein of interest is carried out simultaneously with the formation of the lipid bilayer, since the latter will be included directly in the proteoliposomes.

The production of unilamellar liposomes is described in the literature: reference may be made notably to Donald et al. 2007. To that end, a film formed of the selected lipid composition is first formed on the wall of a glass test tube or flask by evaporation, notably in argon, of the organic solvent in which the lipid mixture was formed. Next, this film is rehydrated with buffer solution, notably buffered to a pH in the range of 6 to 8, ideally of 7.4. These first two steps, for example, are carried out at a temperature in the range of 10 to 35° C., and typically at room temperature (22° C. notably). HEPES or PBS buffer may, for example, be used. Multilamellar vesicles are then obtained and are transformed by repeated cycles of freezing (for example in liquid nitrogen) and of thawing at a temperature of 30 to 40° C., followed ultimately by an extrusion step for adjusting the size of the unilamellar liposomes thus obtained.

The steps of:
a) attaching the tethering peptide onto the surface of the substrate,
b) attaching onto the tethering peptide liposomes comprising a portion of lipids having a chelating polar headgroup enclosing a metal cation, by the establishment of metal-chelate interactions between the metal cation and at least a portion of the histidines located at the C-terminal position,
c) adding a fusogenic agent in order to induce liposome fusion and the formation of a continuous lipid bilayer,
   are, in most cases, carried out at a temperature in the range of 10 to 35° C., and typically at room temperature (22° C. notably) and at atmospheric pressure (in particular at 1013 hPa).

In general, the substrate is placed in a chamber and the various reagents are injected successively with a washing step between each of steps a) to c) with buffer solution, notably buffered to a pH in the range of 6 to 8, ideally of 7.4, thus making it possible to remove all the unbound molecules.

For step a), the substrate is contacted with a buffered aqueous solution of the tethering peptide, typically at a concentration of 10 nM to 10 μM, notably of 25 nM to 4 μM of tethering peptide. The liposomes are then added in the form of a suspension in a buffered aqueous solution (the buffer in which they are formed), to obtain typically in the reaction mixture in contact with the substrate a concentration of 50 to 200 μg/mL. Next, the fusogenic agent is added to obtain typically in the reaction mixture in contact with the substrate a concentration of 10 to 20 μM. The same types of buffer as those described above for preparing unilamellar liposomes may be employed in each of steps a) to c) in the reagent supply solutions.

After formation of the bilayer, it is also possible to add to the reaction medium divalent cations such as calcium (Ca²⁺, etc.), or a chelating agent such as EDTA at a concentration, for example, of 0.5 to 5 mM, with no disturbance of the assembly, i.e., with no detachment of the bilayer after attachment to the tethering peptide being observed.

In conclusion, the invention combines various advantages:
first, the mechanical stability and the robustness of the lipid bilayers attached to the substrate, by reversible linking to a tethering peptide which is itself grafted by covalent bond onto the substrate,
the possibility of adjusting as desired the composition of the lipid bilayer which can thus be adapted to the membrane protein of interest which must be inserted,
the possibility of forming different zones, so as to obtain micro-networks of regions bearing different lipid bilayers and thus the development of biochips having associated membranes,
the possibility of carrying out multiplexed detection without labeling and in real-time by surface plasmon resonance imaging (SPRi) measurement in the case of gold substrates.

The invention thus makes it possible to develop a versatile, high-throughput analysis tool for studying ligand-membrane protein interactions.

The following examples, in reference to the appended Figures, illustrate the invention but are in no way limiting.

FIG. 1 is a schematic representation of an assembly according to the invention.

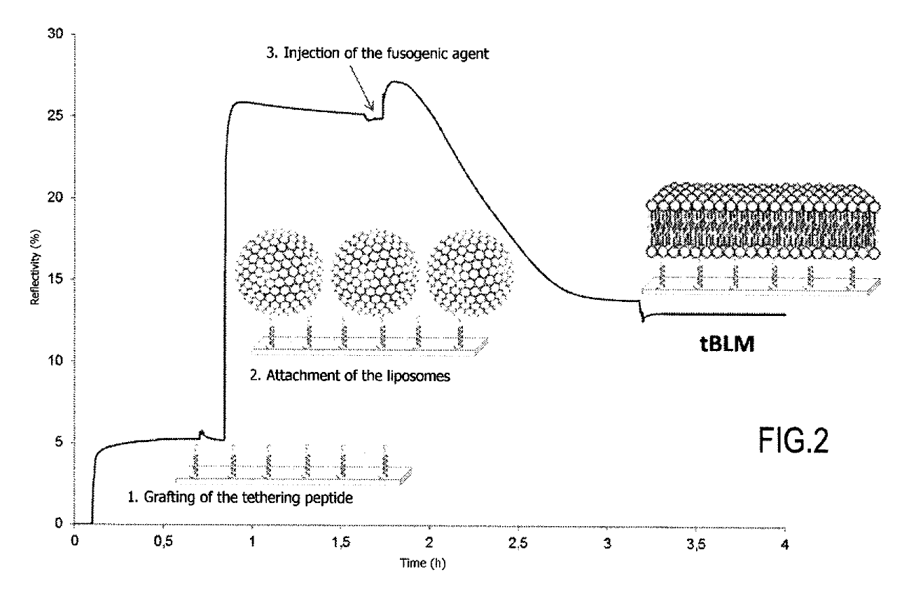

FIG. 2 presents the % reflexivity as a function of time, monitored by surface plasmon resonance imaging (SPRi) in the case of the lipid composition: mixture of DOPC, DOPS and DOGS-NTA-(Ni), in a molar ratio of 74:24:2.

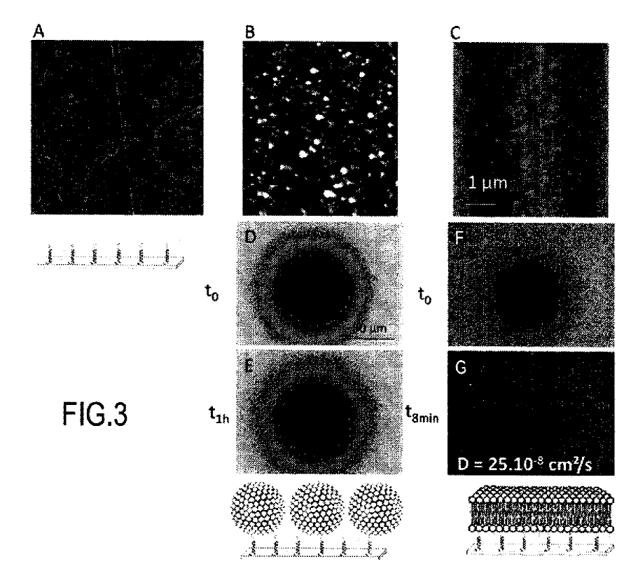

FIG. 3 presents the characterization of the suspended lipid bilayer formation process by atomic force microscopy (AFM) (images A, B and C at top) and by fluorescence recovery after photobleaching (FRAP) (images D, F, E, G at bottom): Top: AFM images of the gold surface after grafting of the tethering peptide (A), after addition of the liposomes containing DOGS-NTA(Ni) (B) and after addition of the fusogenic peptide (C). Bottom: Fluorescence recovery after photobleaching of a surface region covered by non-fused liposomes at 0 min (D) or 1 h after photobleaching (E) and of a surface region after formation of the lipid bilayer by addition of the fusogenic agent at 0 min (F) or 8 min after photobleaching (G).

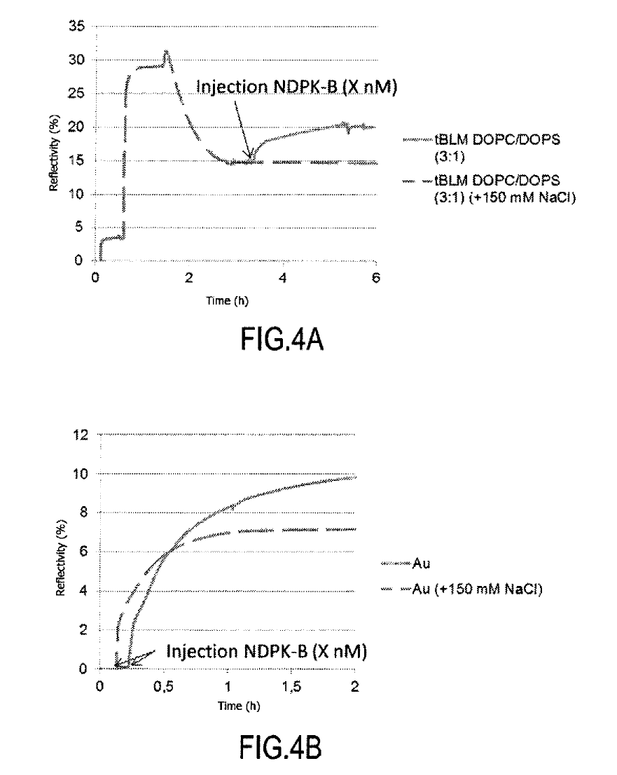

FIG. 4A presents the % reflexivity as a function of time monitored by SPRi of the formation of a lipid bilayer consisting of DOPC/DOPS (3:1) in the presence or the absence of 150 mM NaCl, following interaction of the nucleoside diphosphate kinase B isoform (NDPK-B) at a final concentration of 30 nM. FIG. 4B presents the % reflexivity as a function of time monitored by SPRi of the adsorption of NDPK-B at a final concentration of 30 nM on the gold surface in the presence or the absence of 150 mM NaCl.

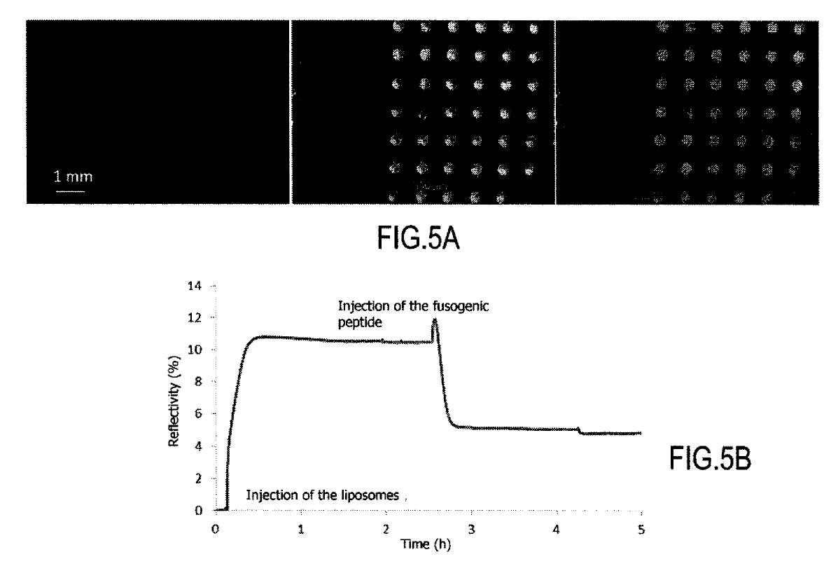

FIG. 5A presents the fluorescence microscopy images of a gold prism after deposition of a tether peptide matrix (image at left) in the case of a biochip format, after incubation with liposomes incorporating 5% of fluorescent probe NBD-PE (middle image) and after liposome fusion (image at right).

FIG. 5B presents the changes in % reflectivity during the lipid bilayer formation process monitored by SPRi for each tethering peptide deposition region, in the case of a biochip format. The baseline corresponds to the signal recorded after deposition of the tethering peptide to the injection of liposomes. Profile representative of all the deposition regions.

### EXAMPLES

### Materials and Reagents

The lipids used come from the company Avanti Polar Lipids®
The SPRi apparatus (SPRi-Lab+) and the prism used (SPRi-Biochip™) corresponding to the gold substrate are marketed by the company Horiba©.
The HEPES buffer and the chloroform come from the company Sigma Aldrich®. The HEPES-NaCl buffer consists of HEPES (10 to 20 mM) and NaCl (0 to 150 mM), and is adjusted to pH 7.4. The PBS buffer consists of phosphate (10 mM), NaCl (140 mM) and KCl (2.7 mM), and is adjusted to pH 7.4.
The Mini-Extruder and the syringes, polycarbonate membranes and prefilters used for preparing the liposomes come from Avanti Polar Lipids®.
The quasi-elastic light scattering (dynamic light scattering, DLS) particle-size analyzer is a Malvern© Zetasizer Nano S.

### ABBREVIATIONS

POPC: 1-palmitoyl-2-oleoyl-sn-glycero-3-phosphocholine

DOPC: 1,2-dioleoyl-glycero-3-phosphocholine

NBD-PE: phosphatidylethanolamine-N-(4-nitrobenzo-2-oxa-1,3-diazole)

PS: phosphatidylserine

PIP₂: phosphatidylinositol-4,5-bisphosphate

DOGS: 1,2-dioleoyl-sn-glycero-3-[(N-(5-amino-1-carboxypentyl)iminodiacetic acid)succinyl]

NTA: nitrilotriacetic acid

POPE: 1-palmitoyl-2-oleoyl-sn-glycero-3-phosphoethanolamine

1. Formation of Liposomes

First, a lipid film is formed on the wall of a glass test tube by evaporation in argon of the organic solvent in which the lipid mixture, prepared in chloroform or in a chloroform/methanol mixture (9:1; v/v) depending on the composition selected, has been deposited. The total quantity of evaporated lipids is 1 mg.

Several lipid compositions were tested:
a mixture of POPC and DOGS-NTA-(Ni), in a molar ratio of 98:2,
a mixture of DOPC and DOGS-NTA-(Ni), in a molar ratio of 98:2,
a mixture of POPC, DOPE-biotin and DOGS-NTA-(Ni), in a molar ratio of 88:10:2,
a mixture of DOPC, DOPS and DOGS-NTA-(Ni), in a molar ratio of 74:24:2,
a mixture of DOPC, DOPS, NBD-PE (fluorescent lipid) and DOGS-NTA-(Ni), in a molar ratio of 70:23:5:2,
a mixture of PC extracted from egg yolk, PS extracted from brain, and DOGS-NTA-(Ni), in a molar ratio of 67:31:2,
a mixture of PC extracted from egg yolk, PS extracted from brain, PIP₂ extracted from brain, and DOGS-NTA-(Ni), in a molar ratio of 67:39:2:2,
a mixture of five lipid components: POPC, sphingomyelin, cholesterol, POPE, DOGS-NTA-(Ni), in a molar ratio of 37:33:19:9:2, which mimics the composition of the outer leaflet of the plasma membrane of eukaryotic cells.

First, a lipid film is formed on the wall of a glass test tube by evaporation in argon of the organic solvent in which the lipid mixture was formed. The lipid film obtained is then hydrated with HEPES-NaCl buffer (pH 7.4) or with PBS buffer in order to have a lipid concentration of 1 mg/mL. This step leads to the separation of the bilayer fragments which will form multilamellar vesicles (MLVs). These MLVs are then transformed by repeated cycles of freezing in liquid nitrogen and of thawing in a 38° C. water bath (6×5 min of freezing and 6×10 min of thawing), which are followed by an extrusion step (21 passes through a 400 nm membrane, then 21 passes through a 100 nm or smaller membrane). These extrusion steps produce large unilamellar vesicles (LUVs) of the desired size (Donald, K. M., L. J. Blum, J. J. Gooding, T. Böcking, A. I. Mechler, A. P. Girard-Egrot, and S. M. Valenzuela. 2007. Nanobiotechnology of Biomimetic Membranes. Langmuir-Blodgett Technique for Synthesis of Biomimetic Lipid Membranes. p. 25-37. Liposome Techniques for Synthesis of Biomimetic Lipid Membranes. p. 77-79).

The size of the liposomes is controlled by quasi-elastic light scattering, also called dynamic light scattering (DLS).

2. Formation of the Lipid Bilayer

To form the bilayer, one first injects into the reaction chamber the peptide in aqueous solution buffered with 10 to 20 mM HEPES buffer (pH 7.4) supplemented with 150 mM NaCl at an initial concentration of 9.74·10⁻⁴ M (or 2.5 mg/mL) in order to obtain a final concentration in the chamber ranging between 25 nM and 4 μM of SEQ ID NO: 1: CSRARKQAASIKVAVSADRHHHH which will serve as the link between the bilayer and the substrate surface and which will come to be attached onto the gold by means of its thiol bonds (N-terminal cysteine). The 4 μM concentration of tethering peptide corresponds to the minimal concentration for obtaining a saturation of the surface with grafted tethering peptide. The liposomes, at the initial lipid concentration of 1 mg/mL, are then injected into the reaction chamber at a final concentration of 100 μg/mL and are grafted onto the peptide. The fusogenic peptide of SEQ ID NO: 2: SGSWLRDVWDWICTVLTDFKTWLQSKWYKD in solution in 10 to 20 mM HEPES buffer (pH 7.4) supplemented with 150 mM NaCl is then injected at a final concentration ranging between 10 and 20 μM. It will interact with the liposomes and allow the passage of LUVs having a planar bilayer. Between each deposition, washes with 10 to 20 mM HEPES working buffer (pH 7.4) supplemented with 150 mM NaCl are carried out (10 times the volume of the reaction chamber), thus making it possible to remove all the molecules which have not been specifically bound.

Tests were carried out by using 25 nM and 4 μM final concentrations of tethering peptide. The adsorption of the tethering peptide onto the gold surface was monitored by surface plasmon resonance. The SPRi apparatus (SPRi-Lab+) and the prism (SPRi-Biochip™) used are marketed by the company Horiba©. Incubation of the gold surface with the solution containing a 4 μM final concentration of tethering peptide induces a 0 to 5% variation of the reflectivity signal, as illustrated in FIG. 2, demonstrating the adsorption of the peptide onto the gold. This variation of reflectivity is the maximum variation which can be obtained when the tethering peptide saturates the surface. It is not modified after intensive rinsing.

The analyses carried out on the percent reflectivity variation values make it possible to determine the density of the molecules grafted onto the surface. This density may be estimated by using the calibration equation of the apparatus used (SPRi-Lab+):

$$Γ = \frac{Δ R \cdot {L}_{ZC}}{{S}_{P , R} \cdot \frac{\partial n}{\partial C}}$$

where ΔR is the variation of reflectivity in percent, L_{ZC} the depth of penetration of the plasmon wave (1.02·10⁻⁴ mm), the δn/δC ratio is set at 1.9·10⁻¹⁰ mm³·pg⁻¹, S_{P,R} is the sensitivity of the SPR in percent per refractive index unit (2.25·10³%·RIU⁻¹) and r corresponds to the density in pmol·mm⁻².

The molecular density determined for a maximum variation of 5% obtained for a 4 μM final concentration of tethering peptide in the reaction chamber and corresponding to the saturation signal is 2.8·10¹³ tethering peptide molecules/cm². At 25 nM, the density is 2·10¹² tethering peptide molecules/cm², leading to a theoretical spacing of 5.8 nm between each tether, if the latter are distributed homogeneously on the substrate.

Liposomes containing 1 to 2% of a lipid having an NTA(Ni) chelating headgroup (18:1 DOGS-NTA(Ni) (1,2-dioleoyl-sn-glycero-3-[(N-(5-amino-1-carboxypentyl)iminodiacetic acid)succinyl])) was used for forming the lipid bilayer. The procedure was tested with liposomes of sizes ranging between 30 and 100 nm and of variable lipid compositions including 98% DOPC, 98% POPC, DOPC/DOPS, POPC/POPS (variable ratios), natural extracts of PC and of PS (variable ratios), in the absence or in the presence of PIP₂ and/or of cholesterol. Addition of liposomes at a 100 μg/mL final concentration of variable lipid compositions containing the chelating lipid DOGS-NTA(Ni) induces a large increase in reflectivity, as illustrated in FIG. 2, which represents a change in the optical properties of the surface of the SPR prism. This reflectivity value is not changed after rinsing. The Ni-NTA chelating headgroup thus interacts specifically with the polyhistidine tag of the peptide, allowing the attachment of liposomes.

Addition of the fusogenic peptide of SEQ ID NO: 2 induces, as shown in FIG. 2, firstly, a slight increase in the reflectivity which represents its insertion into the liposomes, followed, secondly, by a reduction in the signal which was interpreted previously, for liposomes deposited directly onto the gold surface without addition of tethering peptide, as the rupturing of the liposomes and the formation of a homogeneous lipid bilayer on the surface of the prism (Chah and Zare 2008). This characteristic profile, presented in FIG. 2, was obtained with all the lipid compositions listed above.

3. Characterization of the Lipid Bilayer Attached to the Tethering Peptide

The steps for forming the lipid bilayer attached to the tethering peptide, itself linked to the gold surface, were characterized by atomic force microscopy (AFM) (SOLVER-PRO© from NT-MDT® (maximum sweep 50×50×2.5 μm±10%; the AFM tips used are CSG01 from NT-MDT® (stiffness: 0.03 mN·m⁻¹)) and by measurement of fluorescence recovery after photobleaching (FRAP) (Zeiss Observer Z1). FIG. 3 shows the images obtained in the case of a bilayer formed of POPC:DOGS-NTA(Ni) (molar ratio of 98:2) This case was selected to show that the fusogenic peptide used by Cho et al. was as effective for forming membranes on tethers (“study”) as directly on the substrate (“study by Cho et al. without tethers”).

The AFM images of the surface before and after incubation with the tethering peptide (FIG. 3A) show a rough surface with scratches characteristic of commercial gold prisms. After incubation of the prism bearing the tethering peptide with the suspension of unilamellar liposomes, a surface covered with vesicles is observed (FIG. 3B), indicating the adsorption of the liposomes onto the surface. After injection of the fusogenic peptide, the surface appears homogeneous (FIG. 3C), which confirms the fusion of the vesicles onto the surface and the covering of the gold surface by a continuous layer of lipids.

The homogeneity and the fluidity of the lipid bilayer formed were confirmed by fluorescence microscopy and by fluorescence recovery after photobleaching (FRAP). In this case, liposomes containing a fluorescent lipid (5 mol % NBD-PE: 1,2-dioleoyl-sn-glycero-3-phosphoethanolamine-N-(7-nitro-2-1,3-benzoxadiazol-4-yl) ammonium salt) were used. These liposomes are prepared, as described above, by adding 5 mol % NBD-PE. A portion of the surface was exposed to a powerful beam of light focused by the microscope objective, before and after addition of the fusogenic peptide. The fluorophore present in this region undergoes a type of destruction referred to as photobleaching (FIGS. 3D and 3F). If the movement of the fluorophore within the membrane leaflet is not constrained and if the lipid bilayer is fluid and continuous, the fluorescent molecules located in the nearby areas will diffuse freely over time and the photobleached region gradually becomes fluorescent again. When the FRAP measurement is taken before addition of fusogenic peptide, the photobleached zone does not become fluorescent again (FIG. 3E). The fluorophore cannot diffuse on the surface, which is characteristic of a discontinuity due to the vesicle population attached to the surface but not fused. When the measurement is taken after addition of the fusogenic agent, a redistribution of the two populations between the bleached zone and the adjacent medium takes place until the photobleached zone disappears (FIG. 3G). The fluorescent molecule thus diffuses freely on the surface, attesting to the formation of a continuous and homogeneous bilayer.

The analysis of the kinetics of fluorescence recovery makes it possible to deduce the lateral diffusion coefficient “D”, characteristic of the fluidity of the lipid bilayer. A value of 25·10⁻⁸ cm²/s was determined, which corresponds to a lipid bilayer that is more fluid than those described for attachment by means of protein/ligand systems (Rossi et al. 2011).

The integrity of the lipid bilayer formed on the gold surface, in turn, was confirmed by using the nucleoside diphosphate kinase B isoform (NDPK-B). This protein interacts specifically with the PS molecules and this interaction is inhibited in the presence of 150 mM NaCl (François-Moutal et al. 2014). Addition of NDPK-B after formation of a bilayer formed on the tethering peptides consisting of DOPC/DOPS (molar ratio of 3:1) in the absence of NaCl induces an increase in the reflectivity recorded by SPRi, which expresses a specific interaction of the protein with the bilayer. In the presence of 150 mM NaCl, there is no increase (FIG. 4A). It was noted that this protein is absorbed onto the gold surface in the presence and in the absence of 150 mM NaCl (FIG. 4B). The absence of adsorption in the presence of 150 mM NaCl when the surface is covered by the lipid bilayer suspended on the tethering peptides indicates that the gold surface is thoroughly covered and that the lipid bilayer is integral and continuous; this continuity preventing the nonspecific interaction of the protein with the bare gold.

4. Construction of the Bilayer in Biochip Format:

A major challenge in producing lipid membranes on a solid substrate is the possibility of carrying out high-throughput measurements. The conventional methods of automated deposition involving dehydration of the samples cannot be applied to the deposition of lipid vesicles.

According to the protocol described above, the depositions of steps a) to c) of the method according to the invention were carried out using a piezoelectric spotter device (sciFLEXARRAYER S3, SCIENION, Germany) on a gold prism. A tethering peptide (0.04 to 4 ng/spot (2.5·10¹² to 2.5·10¹⁴ molecules/cm²)) was first deposited. The attachment of the liposomes (consisting of DOPC/DOPS, molar ratio of 3:1, incorporating 5% NBD-PE and 2% DOGS-NTA(Ni)) onto the surfaces covered with tethering peptide was monitored by SPRi and by fluorescence microscopy (FIGS. 5A and 5B). After deposition of the liposomes, the regions where the peptide was deposited become fluorescent, which attests to the attachment of the liposomes to the tethering peptides deposited by addressing (FIG. 5—middle image). This step is accompanied by an increase in the reflectivity at each deposition of tethering peptide (FIG. 5B). After addition of the fusogenic agent, fluorescence persists (FIG. 5—image at right) and the reduction in the reflectivity recorded by SPRi (FIG. 5B) attests to the formation of the bilayer suspended above each region of tethering spacer peptide deposited by micro-addressing.

### REFERENCES CITED

Chah, S. and R. N. Zare (2008). “Surface plasmon resonance study of vesicle rupture by virus-mimetic attack.” Physical Chemistry Chemical Physics 10(22): 3203-3208.
Cho, N.-J., S.-J. Cho, K. H. Cheong, J. S. Glenn and C. W. Frank (2007). “Employing an Amphipathic Viral Peptide to Create a Lipid Bilayer on Au and TiO₂.” Journal of the American Chemical Society 129(33): 10050-10051.
Cho, N.-1, G. Wang, M. Edvardsson, J. S. Glenn, F. Hook and C. W. Frank (2009). “Alpha-Helical Peptide-Induced Vesicle Rupture Revealing New Insight into the Vesicle Fusion Process As Monitored in Situ by Quartz Crystal Microbalance-Dissipation and Reflectometry.” Analytical Chemistry 81(12): 4752-4761.
Cho, N.-J., C. W. Frank, B. Kasemo and F. Hook (2010). “Quartz crystal microbalance with dissipation monitoring of supported lipid bilayers on various substrates”. Nature Protocols 5: 1096-1106.
Coutable, A., T. Christophe, J. Chalmeau, J.-M. François, C. Vieu, V. Noireaux, E. Trévisiol, (2014). “Preparation of tethered-lipid bilayers on gold surfaces for the incorporation of integral membrane proteins synthesized by cell-free expression.” Langmuir 30(11): 3132-3141.
Donald, K. M., L. J. Blum, J. J. Gooding, T. Böcking, A. I. Mechler, A. P. Girard-Egrot, and S. M. Valenzuela. (2007) Nanobiotechnology of Biomimetic Membranes. Langmuir-Blodgett Technique for Synthesis of Biomimetic Lipid Membranes. p. 25-37. Liposome Techniques for Synthesis of Biomimetic Lipid Membranes. p. 77-79.
François-Moutal, L., Marcillat O. and Granjon T. (2014). “Structural comparison of highly similar nucleoside diphosphate kinases: molecular explanation of distinct membrane binding behavior.” Biochimie 105:110-118.
Hardy, G. J., R. Nayak, S. Munir Alam, J. G. Shapter, F. Heinrich and S. Zauscher (2012). “Biomimetic supported lipid bilayers with high cholesterol content formed by α-helical peptide-induced vesicle fusion.” Journal of Materials Chemistry 22: 19506-19513.
Lorin A., B. Charloteaux, L. Lins and R. Brasseur (2007) “Implication des peptides de fusion des glycoprotéins de fusion virales de classe i dans la fusion membranaire”, BASE, vol. 11, no. 4, (http://popups.ulg.ac.be/1780-4507/index.php?id=1740).
Robelek, R., E. S. Lemker, B. Wiltschi, V. Kirste, R. Naumann, D. Oesterhelt and E.-K. Sinner (2007). “Incorporation of In Vitro Synthesized GPCR into a Tethered Artificial Lipid Membrane System.” Angewandte Chemie International Edition 46(4): 605-608.
Rossi, C., S. Doumiati, C. Lazzarelli, M. Davi, F. Meddar, D. Ladant and J. Chopineau (2011). “A Tethered Bilayer Assembled on Top of Immobilized Calmodulin to Mimic Cellular Compartmentalization.” Plos One 6(4).
Yldiz, A. A., U. H. Yildiz, B. Liedberg and E. K. Sinner (2013). “Biomimetic membrane platform: Fabrication, characterization and applications.” Colloids and Surfaces B-Biointerfaces 103: 510-516.
Cho N.-J., Cheong K. H., Glen J. S., Frank C. W. U.S. Pat. No. 8,211,712 B2 “Method of Fabricating Lipid Bilayer Membranes On Solid Supports”.

1- Assembly consisting of a substrate on which at least one lipid bilayer is attached by means of a peptide, referred to as the tethering peptide, which is itself linked to the substrate, characterized in that the tethering peptide has a C-terminal end consisting of at least four consecutive histidines and in that the lipid bilayer comprises a portion of lipids having a chelating polar headgroup enclosing a metal cation providing the link with the tethering peptide as a result of metal-chelate interactions between the metal cation and at least a portion of the histidines constituting the C-terminal end of the tethering peptide.

2- Assembly according to claim 1 characterized in that the metal cation is a nickel cation, a gadolinium cation or a copper cation.

3- Assembly according to claim 1 characterized in that the metal cation is a nickel cation and the chelating polar headgroup is nitrilotriacetic acid.

4- Assembly according to claim 1 characterized in that the portion of lipids having a chelating polar headgroup enclosing a metal cation represents from 0.5 to 5 mol %, preferably from 1 to 2 mol %, of the totality of the lipids forming the lipid bilayer.

5- Assembly according to claim 1 characterized in that the tethering peptide is attached covalently to the substrate, preferably by its N-terminal end.

6- Assembly according to claim 1 characterized in that the substrate is made of gold.

7- Assembly according to claim 6 characterized in that the tethering peptide comprises a cysteine at its N-terminal end establishing an S—Au bond with the gold substrate.

8- Assembly according to claim 1 characterized in that the C-terminal end of the tethering peptide consists of four, five or six consecutive histidines.

9- Assembly according to claim 1 characterized in that the link with the tethering peptide is provided as a result of metal-chelate interactions established between the metal cation and two of the histidines located in the C-terminal portion of the tethering peptide.

10- Assembly according to claim 1 characterized in that the composition of the lipid bilayer is selected so as to mimic a biological membrane, and notably a cell membrane.

11- Assembly according to claim 1 characterized in that the lipid bilayer is fluid and continuous.

12- Assembly according to claim 1 characterized in that a membrane protein, preferably an integral membrane protein, is inserted into the lipid bilayer.

13- Assembly according to claim 1 characterized in that the substrate has several zones on which a lipid bilayer is attached by means of a peptide, referred to as the tethering peptide, said tethering peptide having a C-terminal end consisting of at least four consecutive histidines and the lipid bilayer comprising a portion of lipids having a chelating polar headgroup which encloses a metal cation and provides the link with the tethering peptide as a result of metal-chelate interactions between the metal cation and at least a portion of the histidines located in the C-terminal portion of the tethering peptide.

14- Method for preparing an assembly according to claim 1 characterized in that it comprises the following successive steps:
a) attaching the tethering peptide to the surface of the substrate,
b) attaching onto the tethering peptide liposomes comprising a portion of lipids having a chelating polar headgroup enclosing a metal cation, said attaching being carried out by the establishment of metal-chelate interactions between the metal cation and at least a portion of the histidines in the portion at the C-terminal position;
c) adding a fusogenic agent in order to induce liposome fusion and the formation of a continuous lipid bilayer.

15- Preparation method according to claim 14 characterized in that the lipid bilayer is obtained by fusion of unilamellar liposomes having an average diameter ranging between 30 and 500 nm.

16- Preparation method according to claim 14 characterized in that the liposomes are proteoliposomes.

17- Preparation method according to claim 14 characterized in that fusion is obtained by means of a fusogenic peptide, preferably of SEQ ID NO 2.

18- Method of detection by surface plasmon resonance imaging using an assembly according to claim 1 wherein the substrate is made of gold.
